(19) United States
(12) Patent Application Publication
(10) Pub. No.: US 2023/0329653 A1
(43) Pub. Date: Oct. 19, 2023

(54) METHOD AND APPARATUS FOR BREAST IMAGING

(71) Applicant: Shabbir Bakir Bambot, West Hills, CA (US)

(72) Inventor: Shabbir Bakir Bambot, West Hills, CA (US)

(21) Appl. No.: 18/340,337

(22) Filed: Jun. 23, 2023

Related U.S. Application Data
(63) Continuation-in-part of application No. 17/446,079, filed on Aug. 26, 2021, now abandoned.
(60) Provisional application No. 63/194,455, filed on May 28, 2021.
(60) Provisional application No. 63/128,173, filed on Dec. 20, 2020.

(51) Int. Cl. A61B 6/04; A61B 6/00; A61B 6/02

(52) U.S. Cl. CPC A61B 6/0414; A61B 6/502; A61B 6/4417; A61B 6/547; A61B 6/025

(57) ABSTRACT

A method for imaging is provided that may include compressing breast tissue with a compression paddle with a compression surface made of a membranous sheet at a first compression force, obtaining x-ray images of the breast tissue while compressing the breast tissue at the first compression force, varying the first compression force on the breast tissue to provide a second compression force that is different than the first compression force, and obtaining ultrasound images of the breast tissue while compressing the breast tissue at the second compression force.

### RELATED APPLICATION

The present application is a continuation-in-part of U.S. patent application Ser. No. 17/446,079, Titled “METHOD AND APPARATUS FOR BREAST IMAGING” which was filed on Aug. 26, 2021 and claims priority to U.S. Provisional Application No. 63/194,455, Titled “METHOD AND APPARATUS FOR AUTOMATED BREAST ULTRASOUND” which was filed on 28 May 2021, and U.S. Provisional Application No. 63/128,173, Titled “METHOD AND APPARATUS FOR AUTOMATED BREAST ULTRASOUND” which was filed 20 Dec. 2020 the complete subject matter of each which is expressly incorporated herein by reference in their entireties.

### BACKGROUND

The present application relates generally to the field of cancer imaging and diagnosis and, more specifically, to breast cancer imaging wherein ultrasound imaging is performed in the same patient orientation or position and in the same procedure/visit as x-ray mammography or tomosynthesis for improved detection of breast cancer.

Breast cancer is the second most common cancer among women overall and is the most common cause of cancer related death among minority populations in the US. Despite its success in reducing breast cancer mortality, X-ray mammography continues to miss cancer in a significant number of women and particularly in women with dense breast tissue. Dense breast tissue occurs due to high fibroglandular tissue content which has higher x-ray absorption and is therefore not well penetrated by x-rays compared to surrounding fatty tissue. Mammograms from women with dense breast tissue contain little or no information in areas with fibroglandular tissue. A study by Sprague et. al. of over 1.5 million women 40 to 74 years of age titled “Prevalence of mammographically dense breasts in the United States” published in the September 2014 issue of the Journal of the National Cancer Institute, 43.3% of mammograms had heterogeneously or extremely dense breasts. For women aged 40 to 44 years this percentage increased to 57. This means that in these women at least a portion of the breast area on the x-ray mammogram cannot be scrutinized well for lesions by x-ray mammography alone. As a result, lesions camouflaged by dense breast tissue may go undetected. Recognizing this the US FDA now requires mammography facilities to notify patients about the density of their breasts and recommends that patients with dense breasts talk to their health care provider about breast density, risks for breast cancer and their individual situation and the need to pursue supplemental screening beyond mammography.

Ultrasound is the most widely prescribed supplemental screening method. It is widely available, fairly easy to perform, does not expose a person to radiation and is less expensive than other testing options. It is especially useful in women with dense breasts which can hide cancer in a mammogram and ultrasound helps distinguish potentially suspicious solid masses from benign cysts. Thus ultrasound in conjunction with mammography can help better detect early breast cancer. Indeed, in one study a 55% increase in detected breast cancer by physician performed ultrasound of mammography screened patients. Ultrasound is also routinely used to target breast biopsy. Supplemental breast cancer screening using ultrasound can however significantly increase unnecessary patient callbacks and biopsies due to its high false positive rate. For example in the study a significantly lower positive predictive value for ultrasound guided biopsy of about 10% compared to x-ray mammography which was 23%.

Most ultrasound breast exams are done manually by experienced sonographers using handheld transducers. The patient is examined while sitting or supine with an ultrasound transducer manipulated to scan over the breast in a procedure that can take up to 20 minutes. Manual ultrasound scanning provides the best image quality and highest accuracy. However, this is highly dependent upon the ability and knowledge of the sonographer in seeing and recognizing internal tissue features and his or her experience and skill in handling and manipulating the ultrasound transducer on the breast tissue surface to better visualize these features. Such manipulation includes applying pressure, tilting, twisting, and rocking the transducer to change the angle of insertion of ultrasound energy into the tissue in order to improve the visibility of features in the body and to eliminate image artifacts. However, experienced sonographers are scarce and where available there is significant variability in their experience and skill which leads to significant variability in their diagnostic accuracy.

The variability of sonographer skill and experience and availability makes it difficult to standardize breast ultrasound in a manner similar to mammography. Standardization is particularly important for gaining acceptance in a mass-screening environment. In recent years automated breast ultrasound systems (ABUS) have been introduced to help standardize and make breast tissue ultrasound exams operator independent. Examples include ABUS systems such as the GE Invenia™ or prone as in the Hitachi Sofia 3D breast ultrasound system. The breast tissue of the patient, while lying either supine or prone, is automatically scanned with an ultrasound transducer in a system that is pressed against the breast tissue. However ABUS is still conducted on a patient positioned different from mammography. Mammography is performed on an upright patient (transverse image) while breast ultrasound both manual and ABUS is performed on a supine or prone patient (coronal image) which makes image correlation a challenge. This makes correlating findings between the two imaging modalities a challenge and could adversely impact diagnostic performance.

Breast ultrasound today requires a separate procedure and, in most cases, a separate follow up appointment resulting in a patient hand-off to a clinician generally different from the one performing the mammography. The ultrasound viewings are conducted independently on the monitor of the ultrasound machine in real-time without referring to any x-ray mammogram information that may exist for the patient. Information of diagnostic importance is therefore lost or not taken into consideration which could adversely impact diagnostic performance. ABUS systems also require additional dedicated equipment and space which is always at a premium in most clinical facilities. The additional time, equipment and space required to conduct the ultrasound examination can be cost-prohibitive in today's mass screening environment.

In view of the above, acquiring ultrasound imaging data with the patient in the same position for both procedures, i.e. without having to reposition the patient between the two measurement modes, mammography and ultrasound. would provide many benefits. Such a system would provide images of the breast that correspond and allow exact clinical comparison between the two modalities. For example, such images would not require the clinician to recalculate or guess whether findings between the two modalities are in the same location or not. Removing the need for a separate procedure would make it more acceptable to patients and the immediate availability of images from both modalities would make the diagnostic procedure more efficient.

Attempts have been made to combine mammography and breast ultrasound in the same procedure by adding automated breast ultrasound capability onto the breast compression paddle of a mammography system. For example U.S. Pat. Nos. 5,474,072 and 5,479,927, incorporated herein by reference, describe a system in which an ultrasound transducer scans the breast through a rigid mammography compression paddle made of sonolucent material. U.S. Pat. Nos. 6,574,499 and 6,876,879 incorporated herein by reference, describe a similar system and use a motion control system for movement and control of the ultrasound transducer. Unfortunately, the material thickness of the breast compression paddle, needed to provide rigidity for adequate breast compression, significantly attenuates ultrasound energy. Breast ultrasound is typically done using frequencies from 5-20 MHz using transducers that have a total dynamic range of 100 dB. At 10 MHz and a 0.5 to 1 cm round trip path through a typical compression plate, the attenuation in transmission through most polymers would be 10-50 dB. This compromises image quality making it inferior to handheld ultrasound imaging. To mitigate the losses due to transmission attenuation U.S. Pat. No. 9,949,719 incorporated herein as a reference, uses a taut polyester mesh to provide rigidity. This tautness or rigidity however restricts modulating the contact pressure between the transducer and tissue and the transducer's orientation with respect to the tissue surface and hinders access to the areolar, lateral and medial areas of the breast i.e. where the tissue falls away from the compression. Obtaining ultrasound images of these areas is desirable as they frequently harbor cancers. Further these areas are imaged with x-ray mammography and having supplemental ultrasound images of these areas can improve diagnosis.

Another area that is found to frequently harbor breast cancers is the retroglandular area or area near the chest wall. The retroglandular area accounts for almost a third of all cancers found. Location of missed cancers can include Retroglandular area (33%), lateral parenchyma (31%), central (18%), medial (13%) and subareolar (4%). Mammography does not include all tissue in this area even with optimal patient positioning. Obtaining ultrasound images of these areas will complement mammography and make for a more comprehensive test.

There remains a strong unresolved and continuing need for a system and method for fast and efficient ultrasound imaging of the entire breast in the same patient configuration as mammography or tomosynthesis. It is also desirable to acquire images using both modalities in the same procedure and patient encounter and using the same equipment. This will help remove the challenges of co-registration of features seen in either imaging modality and allow both image sets to be reviewed side-by-side thereby significantly improving accuracy of screening and diagnosis. The availability of side-by-side images using two different modalities in the same physical configuration will also open opportunities for additional improvement in diagnosis using artificial intelligence and machine learning. This will also help reduce cost, increase hospital efficiency, improve patient management by removing the need for multiple patient visits and the need for additional and large equipment and space.

### SUMMARY

In accordance with embodiments herein, a method for imaging is provided that can include compressing breast tissue with a compression paddle with a compression surface made of a membranous sheet at a first compression force, obtaining x-ray images of the breast tissue while compressing the breast tissue at the first compression force, varying the first compression force on the breast tissue to provide a second compression force that is different than the first compression force, and obtaining ultrasound images of the breast tissue while compressing the breast tissue at the second compression force.

Optionally, the x-ray images obtained are at least one of planar (2D) images or 2D images for tomosynthesis. In one aspect, obtaining ultrasound images of the breast tissue includes obtaining a plurality of two-dimensional ultrasound images of the breast tissue, and forming a three-dimensional image based on the plurality of two-dimensional images of the breast tissue. In another aspect, the method can also include determining the first compression force with a sensor, and varying the first compression force on the breast tissue to provide the second compression force based on the first compression force determined by the sensor. In one example, the method can also include applying a gel to the membranous sheet prior to obtaining the ultrasound images, and obtaining transverse images of the breast tissue by scanning at least one of the medial margins of the breast tissue or the lateral margins of the breast tissue as a transducer moves between the compression paddle and a breast plate.

Optionally, obtaining the ultrasound images includes mechanically or electrically driving an ultrasound transducer along the membranous sheet to obtain ultrasound image data. In one aspect, the ultrasound images are based on the ultrasound image data. In another aspect, obtaining the ultrasound images includes providing more than one pass over the breast tissue with an ultrasound transducer while compressing the breast tissue at the second compression. In one example, obtaining ultrasound images of the breast tissue while compressing the breast tissue at the second compression force can include scanning the breast tissue with a transducer that dynamically conforms to the breast tissue curvature. In another example, the first compression force can be greater than 20-pounds-force and the second compression force can be less than 20-pounds-force. In yet another example, varying the first compression force on the breast tissue to provide a second compression force that is different than the first compression force can include moving a tensioning lever of compression mechanism from a first position to a second position.

In accordance with embodiments herein a mammogram system for imaging breast tissue is provided that can include a flexible transducer assembly configured to dynamically change shape based on a curvature of breast tissue of a patient.

Optionally, the flexible transducer assembly can include a first transducer section coupled with a hinge to a second transducer section to rotate about the hinge based on the curvature of the breast tissue. In one aspect, the flexible transducer assembly can include a plurality of capacitive micromachined ultrasound transducers formed onto a flexible substrate. In another aspect, the flexible substrate can be a polymide formed using lithography. In one example, the flexible transducer assembly can be configured to obtain data from a retroglandular area of the breast tissue.

In accordance with embodiments herein a compression assembly for compressing breast tissue during imaging is provided that can include a compression paddle with a compression surface made of a membranous sheet and configured to compress breast tissue, and a compression mechanism coupled to the compression paddle and configured to vary tension of the membranous sheet so that the membranous sheet exerts a first compression force on the breast tissue at a first position of the compression mechanism and to compress the breast tissue at a second compression force that is different than the first compression force at a second position of the compression mechanism.

Optionally, the membranous sheet is formed from at least one of biaxially oriented polyethylene (BOP), biaxially oriented polypropylene (BOPP), or biaxially oriented polyethylene terephthalate (BoPET). In one aspect, the membranous sheet can be less than 50 microns in thickness. In another aspect, the compression mechanism can include at least one tensioning lever that is received within a hook of the compression surface, the at least one tensioning lever configured to move from the first position to the second position. In one example, the first compression force can be greater than 19-pounds-force and the second compression force is less than 16-pounds-force.

### BRIEF DESCRIPTION OF THE DRAWINGS

FIG. 1 is a perspective view of an ultrasound scanning system in accordance with one embodiment of the present disclosure.

FIG. 2A is a schematic diagram of an ultrasound scanning system scanning a breast in accordance with one embodiment of the present disclosure.

FIG. 2B is a schematic diagram of an ultrasound scanning system scanning a breast in accordance with one embodiment of the present disclosure.

FIG. 3A is a schematic diagram of an ultrasound scanning system in accordance with one embodiment of the present disclosure.

FIG. 3B is a schematic diagram of an ultrasound scanning system in accordance with one embodiment of the present disclosure.

FIG. 3C is a schematic diagram of an ultrasound scanning system in accordance with one embodiment of the present disclosure.

FIG. 4A is a schematic diagram of six degrees of freedom in which an ultrasound scanning system can move in accordance with one embodiment of the present disclosure.

FIG. 4B is a schematic diagram of planes in which an ultrasound scanning system can move in accordance with one embodiment of the present disclosure.

FIG. 5 is a schematic flow block diagram of a method of using a combined mammography/tomosynthesis and ultrasound breast system, in accordance with one embodiment of the present disclosure.

FIG. 6 is a schematic diagram of a scan protocol of an ultrasound scanning system in accordance with one embodiment of the present disclosure.

FIG. 7A is a schematic diagram of an ultrasound scanning system in accordance with one embodiment of the present disclosure.

FIG. 7B is a schematic diagram of an ultrasound scanning system in accordance with one embodiment of the present disclosure.

FIG. 8 illustrates a schematic flow block diagram for a method for obtaining ultrasound scans in accordance with one embodiment of the present disclosure.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

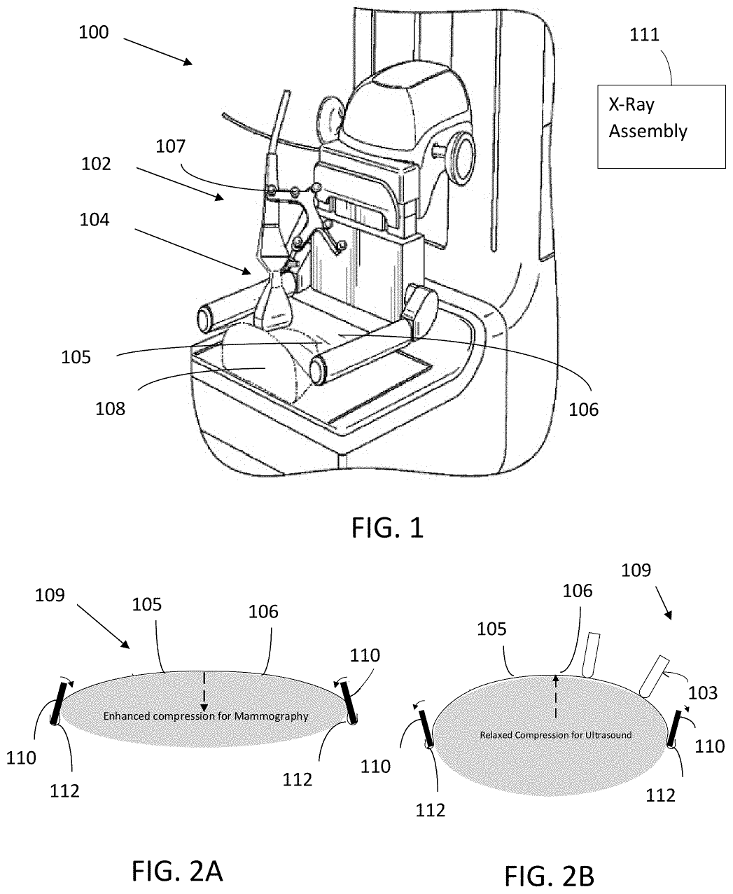

FIGS. 1-8 illustrate alternative embodiments of imaging systems. In particular, the imaging systems illustrated are a multi-modality cancer screening and diagnosis systems that allow cancer screening and diagnosis of a patient using at least two different and sequential imaging techniques. In one embodiment, the object to be imaged is a patient's breast, while in other embodiments other body part may be imaged. Using the imaging system the breast tissue is positioned during the cancer screening and diagnosis procedure on a breast plate of the mammography system where the breast is held and compressed using a compression paddle. One imaging technique that can be utilized is mammography, tomosynthesis (so called three-dimensional mammography) or computed tomography which produces respectively, a planar image, a series of planar images or a three-dimensional x-ray image of the breast. The other imaging technique that may be utilized is three-dimensional ultrasound accomplished by scanning the breast with an ultrasound transducer having a transmitter mounted thereon. The transducer generates planar (two dimensional) brightness mode or B-mode ultrasound images representing slices of tissue that can be generally orthogonal to the scanned surface. The transmitter provides position information for each planar image or slice in six degrees of freedom i.e. the 3 dimensions X, Y and Z and rotation in each dimension (pitch, yaw and roll). The planar images tagged with their positional information can then be used to reconstruct a three-dimensional ultrasound image of the breast.

In addition, the imaging system provides a means for quickly and easily changing the amount of breast compression between the two imaging modalities is provided. The compression paddle can be designed to position and facilitate visualization of the breast, comfortably and without significant movement, for screening or diagnosis with both imaging modalities. Mammography requires vigorous breast compression in order to minimize radiation dose and help prevent patient movement. This is uncomfortable and painful for the patient. Fortunately capturing a mammography image takes only a few seconds. This includes the time taken by the nurse, after positioning the patient, to walk to the console located behind an x-ray protective barrier. Ultrasound scanning, either manual or automated, takes a longer time to scan the breast. Compression for dose reduction is not needed here, and a more relaxed compression to hold the breast in place can be better tolerated and no longer painful for the patient. Further, the clinician can stand with the patient to ensure positioning during the ultrasound scanning procedure since ultrasound does not use ionizing radiation. Therefore the imaging system can be equipped with a means for quickly and easily reducing compression during ultrasound imaging without requiring the patient to move or change position.

Yet another embodiment, the imaging system can provide a means for accessing and scanning the areolar, lateral and medial areas of the breast i.e. where the tissue falls away from the compression. Access to these areas can become more difficult with reduced compression and thickness increases. It is important to have a compressing means such as a film that tightly follows the breast curvature and eliminates air gaps that would adversely affect ultrasound transduction into the tissue. Furthermore, the transducer itself must properly follow the breast curvature and remain orthogonal to the surface at all points along its travel while performing the scan. Staying orthogonal to the tissue surface ensures proper ultrasound energy transduction into and from the tissue. Meanwhile a tracker can report the position of the transducer at all points of the scan so that the exact location, in all six degrees of freedom, is tagged onto the planar image obtained by the transducer to enable reconstruction of the three-dimensional volume image of the breast.

In another embodiment, the imaging system can use a position tracker mounted on an ultrasound transducer that is used for scanning. Using a position tracking method enables 3D reconstruction of the scan volume without restricting the orientation of the transducer and allowing it to freely follow the breast curvature and remain orthogonal to the surface at all points along its travel while performing the scan. It also permits an accounting of areas scanned with a sufficient dwell time for adequate measurement and areas that may have been missed so that the user can be alerted to go back there and complete the scan. Areas missed are indicated by missing voxel values in the 3D reconstructed image or image areas deemed to have an insufficient signal to noise ratio (SNR) likely due to insufficient dwell time. Similarly the transducer must be allowed the flexibility of modulating the local pressure which is necessary to better visualize features in tissue. This flexibility combined with the flexibility afforded by advanced algorithms allows reconstructing more detailed and accurate 3D images of the breast for better detection and diagnosis of abnormalities, particularly in difficult-to-image areas. Transducers used for breast ultrasound are typically 3 to 5 cm in length which is also known as the field-of-view. Therefore completing a scan may require multiple passes over the breast to capture the entire breast volume. In one example, to expedite the scan a longer transducer ranging from 10 to 20 cm may be used. However a linear transducer would provide inadequate energy coupling over the curved breast surface because of its inability to follow the breast curvature and remain orthogonal to the surface at all points along its travel while performing the scan. This can be accomplished using a transducer that can dynamically change curvature. An example of such a transducer is a hinged sectional transducer with two or more sections. Alternatively a flexible transducer can be made using CMUT (Capacitive micromachined ultrasound transducers) technology where transducer elements are formed onto a flexible substrate such as polyimide using lithography. A positive pressure on the transducer against the breast surface can be used to help the transducer maintain contact with and conform to the breast curvature at all points along the scan length.

In another embodiment, a method of examining the tissue at or near the chest wall using the imaging system can be provided. This area is generally excluded from the x-ray mammography image but is found to harbor breast cancers in a significant number of women. Ultrasound uses non-ionizing radiation so there are no safety concerns regarding its use for examining areas near the chest. A method for extending the scan area to include areas of tissue near the chest wall as well as the axilla are provided.

In another example, the imaging system presents a representative ultrasound image that is derived from the three-dimensional reconstructed ultrasound image alongside the mammography image. Mammography produces generally transverse images of the breast in both the cranio-caudal and medio lateral oblique (CC and MLO) positions. Presenting transverse ultrasound images can permit easy comparison and correlation of findings between the two modes.

Transverse images can be obtained by simply taking transverse slices of the reconstructed three-dimensional ultrasound image of the breast. Alternatively, a transverse slice averaged over a user selectable tissue thickness can be presented. The user selectable tissue thickness can vary from a few millimeters to the entire compressed breast thickness that is held between the compression paddle and breast plate of the mammography system.

In another example, reconstructed three-dimensional breast image can have enhanced spatial resolution in the coronal (or sagittal depending upon scan direction) plane matching the resolution of the planar images captured by the ultrasound transducer. However, due to reconstruction artifacts the resolution in the transverse plane may be lower than desirable. Artifacts can result from missing voxel data due to limited dwell times at any given location or from transducer tilting or rocking during a scan resulting in voxels (areas) within the tissue to be missed. In this case the missing voxel is assigned a value derived from that of its nearest neighbors by the reconstruction algorithm. Optionally the user can be asked to re-image those areas marked as having insufficient data. Artifacts can also occur due to multiple values being recorded for a given voxel in which case the reconstruction algorithm must assign it a value derived from the multiple values. Transverse resolution may be improved by taking redundant scans of the tissue which will help “fill in” missing voxels values. It can also be improved by performing both sagittal and coronal scans. The transducer generates planar (two dimensional) images representing slices of tissue that are preferably orthogonal to the scanned surface and the transmitter provides position information is six degrees of freedom for each slice. The six degrees of freedom are the 3 dimensions X, Y and Z and rotation in each dimension (pitch, yaw and roll). The planar images tagged with their positional information can then be used to reconstruct a three-dimensional ultrasound image of the breast.

In sum, the present imaging system and method are directed towards a system for automated or semi-automated ultrasound scanning of tissue such as the human breast to standardize the procedure, improve efficiency and reduce reliance on skilled clinicians and sonographers who perform manual ultrasound scans. The scanning may be done as part of a multi-modality imaging system, such as a combined tomosynthesis and ultrasound imaging system. For example, in such an embodiment, the breast is scanned while under compression with the patient standing to provide transverse views of the breast or more particularly CC or MLO views (cranio-caudal or medio-lateral-oblique views) as in the views generated by mammography. The ultrasound components may be positioned and configured so as not to interfere with or compromise the mammography or tomosynthesis imaging. Further, the ultrasound transducer and associated components may be configured to scan the breast under compression, such as under the compression provided by the compression paddle or assembly used in mammography, so that the acquired image data sets from each modality may be more easily associated with each other due to the conformity of the breast position between the two separate imaging operations. Thus, in such combined embodiments, the mammography and ultrasound image data may be obtained sequentially (or otherwise close in time) in one patient setting after the patient has been prepared, without having to move or reposition the patient.

Though certain of the present embodiments discussed herein are provided in the context of a combined imaging system (such as a combined ultrasound and mammography imaging system) it should be appreciated that such examples are provided for illustration and explanation only and are not intended to be limiting. In particular, certain aspects of the present ultrasound imaging approach may be implemented in imaging contexts that only involve the acquisition of ultrasound image data or, in other contexts, involve the acquisition of ultrasound image data in conjunction with other types of image data than those discussed herein (e.g., mammography image data). Thus, it should be understood and appreciated that the present examples are selected and presented so as to facilitate explanation of the present approach but are not intended to be exhaustive or limiting as to the scope of possible implementations.

With this in mind and turning to FIG. 1 which shows a perspective view of a mammography system 100 that utilizes an ultrasound scanning assembly 102 with a transducer 104 according to an embodiment. The breast compression paddle 105 with a membranous sheet 106 can be used for breast compression for the x-ray mammography procedure as well as for the ultrasound scanning procedure. In one example, the membranous sheet is tautly stretched to provide a dynamically changing pressure to the breast tissue based on the curvature of the breast. In this example the ultrasound scanning procedure utilizes the ultrasound transducer 104 with an optical tracker 107 mounted on the ultrasound transducer. In addition to the ultrasound scanning assembly, the mammography system 100 can include an x-ray assembly 111.

The mammography system 100 includes a breast tissue compression assembly 109 that has the compression paddle 105 that includes and supports the membranous sheet 106 that represents a compression surface that is disposed across a bottom opening thereof and that compresses the breast tissue 108. According to one embodiment the membranous sheet of the compression surface 106 can be used to provide the compression needed for mammography (x-ray scanning) as well as for the ultrasound scanning. For example a higher level of compression of typically about 20-pound force may be required for mammography in order to minimize dose and this compression level may also be suitable for ultrasound scanning. To this end, a compression mechanism (see FIGS. 2A-B) may be coupled to the compression paddle 105 to vary the compression force of the membranous sheet 106 from a first compression force to a second compression force that is different than the first compression force. In one example the first compression force is at or greater than 20-pound force and the second compression force is less than 20-pound force. In another example, the first compression force is greater than 18-pound force while the second compression force is less than 10-pound force.

The breast tissue compression assembly 109 can be equipped with the compression mechanism to relax the compression for ultrasound scanning by releasing the tension on the membranous sheet 106 as shown in FIG. 2A-B to a level more comfortable for the patient. Ultrasound scanning, either manual or automated, takes a longer time to scan the breast tissue. Compression for dose reduction is not needed here and a more relaxed compression to hold the breast tissue in place can be better tolerated and no longer painful for the patient. The membranous material of the membranous sheet 106 that forms the compression surface can be made of a thin plastic or polymer for example a biaxially oriented polyethylene (BOP), biaxially oriented polypropylene (BOPP) or biaxially oriented polyethylene terephthalate (BoPET) which allow extremely thin membranes (approximately 5 to 50 microns) in order to limit ultrasound transduction loss while providing a high degree of tensile strength, biocompatibility and resistance to chemicals, moisture and heat. The ultrasound transducer 104 may contain an ultrasound processor including beamforming electronics, and other processors and equipped with means for wired or wireless communication with a device control, data processing and display device. The scanning procedure itself may be done manually, semi automatically or automatically using a motor device or a robotic arm in accordance with a scan protocol that ensures all areas of the breast tissue are measured using a sufficient dwell time at any given location to obtain an acceptable image.

FIG. 2A-2B show a breast tissue compression assembly 109 that includes the membranous sheet 106. The breast tissue compression assembly 109 in this embodiment includes tensioning levers 110 that are received within a coupling member 112 such as a hook of the membranous sheet 106 that controls the tautness of the membranous material providing the compression force. The tensioning levers 110 can move to multiple positions to vary the tautness of the membranous sheet. In this manner, the tautness can be adjusted for the individual patient. In this manner, when the tensioning levers 110 are in a first position (FIG. 2A) tension, or first compression force, is applied to the compression surface 106 to compress the breast tissue 108 under the compression surface 106. The position of tensioning levers 110 can then be moved to a second position (FIG. 2B) to release the tension, or provide a second compression force, on the compression surface 106 for easy and quick compression relaxation of compression in preparation for ultrasound scanning. As indicated above, while a first and second position are illustrated in the Figures, additional positions between the illustrated first position and second position are additionally provided. In the example, the first compression force is greater than the second compression force as a result of the compression mechanism.

The tensioning levers 110 can be located on either side of the compression paddle 105 and the compression paddle 105 may be mechanically connected to the tensioning levers 110 to stretch the compressing membrane over the breast tissue. These tensioning levers 110 can be moved in preparation for ultrasound scanning in order to release the tension thereby reducing the compression force on the breast tissue 108 while still holding the breast tissue 108 under a relaxed compression force that is lower than the 20-pound force typically required for mammography.

While in the example the compression mechanism includes tensioning levers, in other example embodiments the compression mechanism may include a motor coupled to the membranous sheet to pull the membranous sheet taught. Alternatively, the membranous sheet may be coupled to a roller, lever, or the like to pull the membranous sheet such that a first compression force and second compression force are applied to the breast tissue 108.

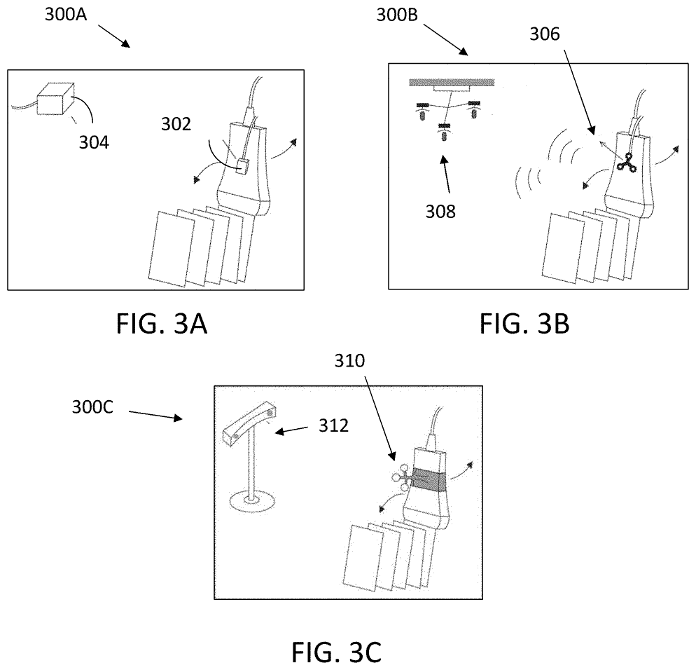

Turning now to FIGS. 3A-3C, each shows various ultrasound scanning assemblies 300A, 300B, 300C that can be utilized for obtaining position information for each scanned 2D image that can allow reconstruction of a 3D volumetric image. The ultrasound scanning assembly 300A of FIG. 3A presents an ultrasound scanning assembly 300A that utilizes a magnetic field sensor 302 along with a spaced magnetic transmitter 304 to obtain the position information. In the embodiment of FIG. 3B, the ultrasound scanning assembly 300B includes sound emitters 306 and spaced microphones 308 that detect the sound from the sound emitters 306. In the embodiment of FIG. 3C the ultrasound scanning assembly 300C has a tracker 310 and spaced cameras 312 that detect signals from the tracker to obtain the positioning information. In each example, position information for each scanned 2D image can be determined accordingly.

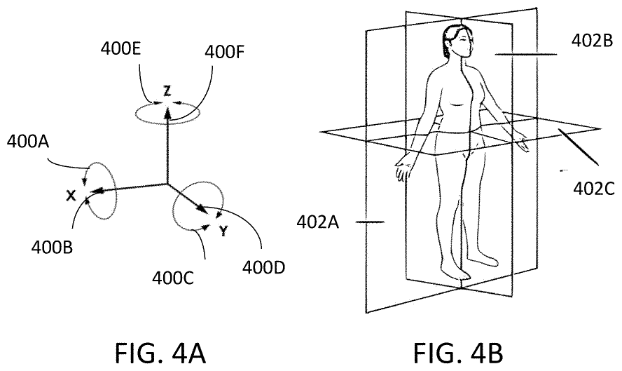

The position information can then be utilized to produce a 3D volumetric image. Because most ultrasound scanning assemblies provide a video output of image frames (2D images) it is necessary to accurately assign each image frame of this video a position, preferably in all 6 degrees of freedom 400A-F as illustrated in FIG. 4A, so that an accurate reconstruction can be performed. Reconstruction involves inserting the collected 2D images into a predefined volumetric grid to produce a 3D volumetric image of the breast tissue that can then be sliced in any plane 402A-C (FIG. 4B) for visualization such as is done on multiplanar image viewers. For example the 3D volumetric image may be sliced for viewing in either the coronal 402A, sagittal 402B or transverse 402C planes. The thickness of each plane 402A-C presented for viewing may be adjusted to represent either a single voxel, a summation of voxels over a selected thickness or a summation of the entire breast tissue thickness in the chosen plane. The reconstruction process involves several steps and can be performed using a variety of different advanced reconstruction algorithms that have been described in the literature, for example in “Huang Q, Zeng Z. A Review of Real-Time 3D Ultrasound Imaging Technology. Biomed Res Int. 2017, Mar. 26” incorporated herein by reference.

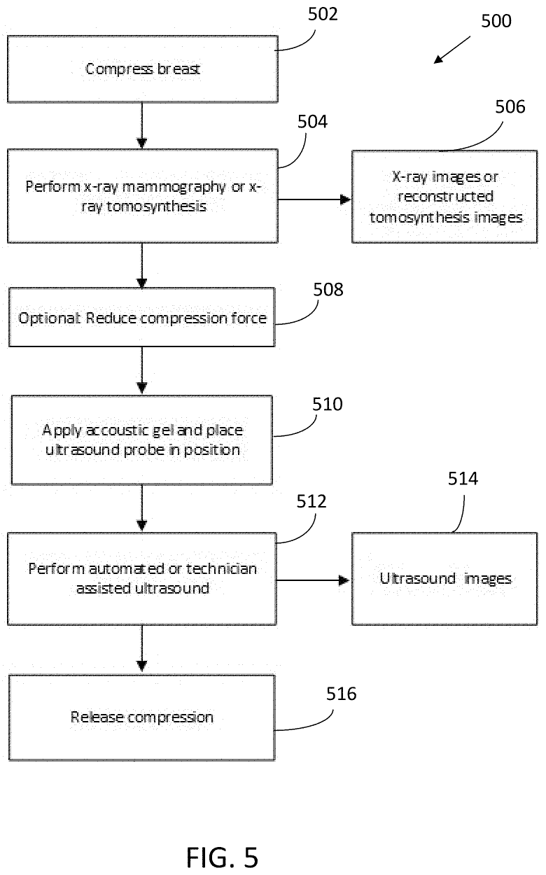

Turning now to FIG. 5 where an example method 500 of multimodality mammography and ultrasound imaging is illustrated in a flow chart. In this example, at 502 the patient is positioned before a mammography system and her breast tissue is compressed to the desired thickness as required for x-ray imaging. In one example, the imaging system and components of imaging system as described in detail in any of the previous FIGS. 1-4C can be utilized to compress the breast tissue and accomplish the steps of the method.

At 504, x-ray imaging is initiated by a clinician from behind an x-ray protective barrier. The x-ray imaging can take anywhere from about one second to a few seconds to complete depending on whether a single image or multiple images are taken. To this end, at 506 a determination is made whether x-ray images or reconstructed images for tomosynthesis are obtained and utilized. Regardless of the type of images obtained, the images are transferred for further processing as needed.

At 508, following x-ray imaging the compression can optionally be relaxed as noted above in preparation for ultrasound scanning. The x-ray clinician can remain beside the patient during the ultrasound scan procedure given that it does not require ionizing radiation. Compression can be performed with a breast tissue compression paddle with a compression surface made of a membranous sheet. In certain embodiments, feedback on compression force measured by sensors or other indicators may be used to determine when sufficient compression is achieved and may be stopped, i.e., when sufficient contact is established. For example, in one embodiment, compression of the breast tissue may be based on a threshold criterion. In another embodiment compression may be determined based on feedback from the patient or the technician.

At 510, ultrasound gel is applied on the membranous sheet compressing the breast tissue in order to reduce the mismatch of acoustic impedance between the ultrasound transducer and skin. In one embodiment, the membranous sheet may be pre-coated with ultrasound gel. For example, membranous sheets coated on both surfaces with polypropylene glycol (PPG), a common ingredient in ultrasound gels, may be used for this purpose and such a material is transparent to x-rays as needed during the mammography procedure and provide the energy coupling needed during ultrasound scanning.

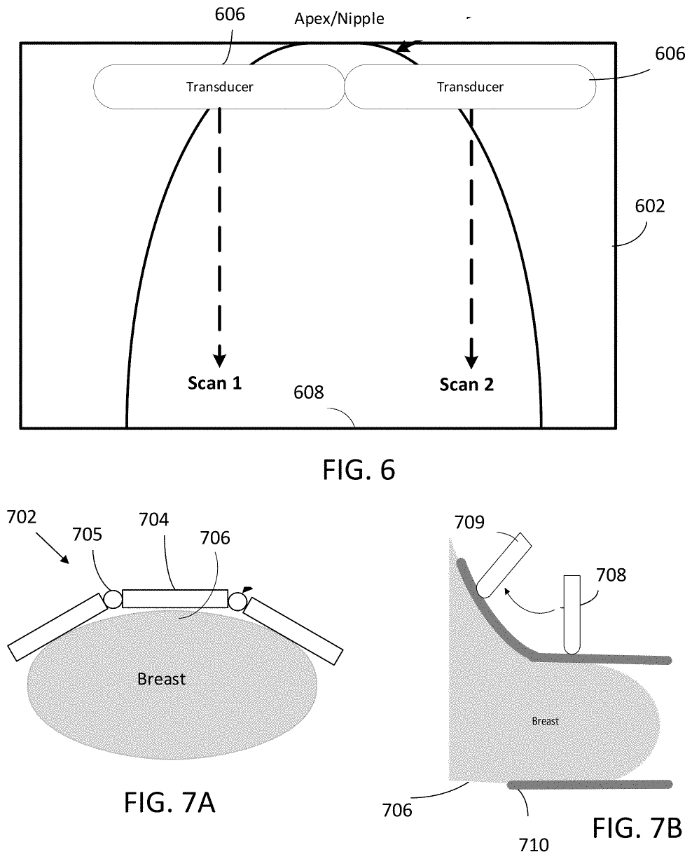

At 512, once positioned, the ultrasound transducer may be mechanically or electromechanically driven along a defined path and may, while driven, acquire ultrasound image data of the underlying breast tissue at 514. In certain embodiments a technician holds the transducer and controls the transducer compression and orientation while scanning the breast tissue. In other embodiments the transducer is held and controlled using automated or robotic means. The ultrasound scan of the breast tissue can be conducted in one or more passes over the breast tissue surface as shown in FIG. 6. In particular, FIG. 6 illustrates the breast tissue paddle 602 compressing the breast tissue 604 between the chest wall 608 and apex/nipple while transducers 606 scan the breast tissue 604 for multiple iterations.

In one embodiment the scan may be conducted in a medial-lateral direction while in another embodiment it may be conducted in an anterior-posterior direction. In certain embodiments, an automated ultrasound scanning system may be used to implement a pre-programmed scan protocol. Such scans may be performed by moving an ultrasound transducer across the breast tissue of a patient without user intervention or guidance during the scan operation. Following ultrasound imaging, at 516 the compression is released.

In addition, once the ultrasound images are obtained, the images can be analyzed to provide an accounting of areas scanned with a sufficient dwell time for adequate measurement and areas that may have been missed so that the user can be alerted to go back there and complete the scan. Areas missed are indicated by missing voxel values in the 3D reconstructed image or image areas deemed to have an insufficient signal to noise ratio (SNR) due to insufficient dwell time.

In all, during the process, breast tissue is compressed with the help of a compression assembly described above and placed into the field of view and maintained in this position. One or more x-ray mammography images are acquired of the breast tissue. The membranous sheet can be pulled taught in a first position during the x-ray imaging to provide desired pressure and is brought to a second position for the ultrasound imaging. In certain embodiments, the ultrasound transducer is at least 15 cm long (such as 19 cm to 30 cm long) to span the entire breast tissue in one pass of the transducer. Alternatively, in another embodiment, two or more ultrasound transducers may be lined up (hingedly arranged) so as to form a flexible and longer transducer length. An advantage of this is that the angle between the transducers may be altered to better contact the curved breast surface. In addition, in certain embodiments, the ultrasound transducer may have surfaces curved to better match the curvature of the breast. In addition, as discussed herein, the ultrasound transducer may be configured for a fast readout, such that the ultrasound scan can be performed in a minute or less.

FIG. 7A illustrates a flexible transducer 702 utilizing sectional transducers in accordance with another embodiment wherein the flexible transducer 702 has two or more hinged sections 704 coupled at hinges 705 that provide the flexibility to adapt to the breast tissue curvature 706 at the scan position. Prior art such as in U.S. Pat. No. 9,808,224 incorporated herein by reference describes a curved transducer to better match breast tissue curvature during a scan. However a fixed curvature such as described there may not match all patients nor meet the curvature of the breast tissue in the same patient at all scan positions. Therefore transducer 702 includes a flexible curvature. Because the transducer is flexible the transducer is allowed to freely follow the breast curvature and remain orthogonal to the surface at all points along its travel while performing the scan. Similarly, the transducer is allowed the flexibility of modulating the local pressure which is necessary to better visualize features in tissue.

As an alternative to a hinged transducer, a flexible transducer can be made using CMUT (Capacitive micromachined ultrasound transducers) technology where transducer elements are formed onto a flexible substrate such as polyimide using lithography. This allows the transducer to conform to the breast tissue curvature of all patients and at all points along the scan length. FIG. 7B illustrates a breast compression paddle with an extension towards the patient's neck area to allow scanning of areas near the chest wall. The scan path for the transducer is designed to include this area moving from position 708 which is within the area imaged in mammography to position 709 the area at or near the chest wall which is often not imaged by mammography even with optimal patient positioning and is found to frequently harbor breast cancers. As a result, a method can be provided for examining the tissue adjacent the chest wall using the imaging system. Adjacent areas include area at the chest wall an by the chest wall. This area is generally excluded from the x-ray mammography image but is found to harbor breast cancers in a significant number of women. Ultrasound uses non-ionizing radiation so there are no safety concerns regarding its use for examining areas near the chest. A method for extending the scan area to include areas of tissue near the chest wall as well as the axilla are provided.

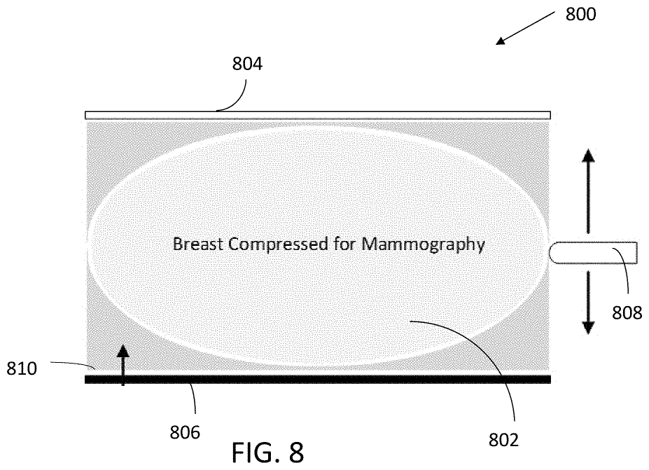

FIG. 8 illustrates an imaging system 800 for directly obtaining transverse images of the breast tissue 802 by scanning from either the medial or lateral margins as shown according to yet another embodiment. In this embodiment, the compression paddle 804 compresses the breast tissue 802 against a breast plate 806 and the transducer 808 can be moved between the compression paddle 804 and breast plate 806 accordingly. In addition, to facilitate obtaining the images, a gel 810 can be utilized between the compression paddle 804 and the breast plate 806 to obtain the traverse ultrasound images. In this manner, a method of examining the tissue at or near the chest wall using the imaging system can be provided. This area is generally excluded from the x-ray mammography image but is found to harbor breast cancers in a significant number of women. Ultrasound uses non-ionizing radiation so there are no safety concerns regarding its use for examining areas near the chest. A method for extending the scan area to include areas of tissue near the chest wall as well as the axilla are provided.

As also discussed above, 3D reconstruction from the two dimensional sagittal or coronal images have a spatial resolution in those planes that is superior to the resolution in the transverse plane. Moreover, transverse images can be used directly, without requiring a 3D reconstruction process, for side-by-side presentation with the transverse images that are generated by mammography. However, acquiring transverse images can be difficult due to the limited space for scanning with an ultrasound transducer around the medial and lateral margins of the breast tissue. This is addressed by surrounding the compressed breast tissue with impedance matching gel or fluid around the medial and lateral margins of the breast tissue.

### CLOSING STATEMENTS

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method, or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random-access memory (RAM), a dynamic random-access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices, and program products according to various example embodiments. These program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A method for imaging comprising:
compressing breast tissue with a compression paddle with a compression surface made of a membranous sheet at a first compression force;
obtaining x-ray images of the breast tissue while compressing the breast tissue at the first compression force;
varying the first compression force on the breast tissue to provide a second compression force that is different than the first compression force; and
obtaining ultrasound images of the breast tissue while compressing the breast tissue at the second compression force.

2. The method of claim 1, wherein the x-ray images obtained are at least one of planar (2D) images or 2D images for tomosynthesis.

3. The method of claim 1, wherein obtaining ultrasound images of the breast tissue includes obtaining a plurality of two-dimensional ultrasound images of the breast tissue, and forming a three-dimensional image based on the plurality of two-dimensional images of the breast tissue.

4. The method of claim 1, further comprising:
determining the first compression force with a sensor; and
varying the first compression force on the breast tissue to provide the second compression force based on the first compression force determined by the sensor.

5. The method of claim 1, further comprising:
applying a gel to the membranous sheet prior to obtaining the ultrasound images; and
obtaining transverse images of the breast tissue by scanning at least one of the medial margins of the breast tissue or the lateral margins of the breast tissue as a transducer moves between the compression paddle and a breast plate.

6. The method of claim 1, wherein obtaining the ultrasound images includes mechanically or electrically driving an ultrasound transducer along the membranous sheet to obtain ultrasound image data.

7. The method of claim 6, wherein the ultrasound images are based on the ultrasound image data.

8. The method of claim 1, wherein obtaining the ultrasound images includes providing more than one pass over the breast tissue with an ultrasound transducer while compressing the breast tissue at the second compression.

9. The method of claim 1 wherein obtaining ultrasound images of the breast tissue while compressing the breast tissue at the second compression force includes scanning the breast tissue with a transducer that dynamically conforms to the breast tissue curvature.

10. The method of claim 1, wherein the first compression force is at or greater than 20-pounds-force and the second compression force is less than 20-pounds-force.

11. The method of claim 1, wherein varying the first compression force on the breast tissue to provide a second compression force that is different than the first compression force includes moving a tensioning lever of compression mechanism from a first position to a second position.

12. The method of claim 1, further comprising:
determining missing voxel values or signal to noise ratio below a threshold of the ultrasound images obtained for scanned areas; and
alerting a user of the scanned areas wherein the missing voxel values are presented or the signal to noise ratio is below the threshold.

13. A mammogram system for imaging breast tissue comprising:
a flexible transducer assembly configured to dynamically change shape based on a curvature of breast tissue of a patient.

14. The mammogram system of claim 13, wherein the flexible transducer assembly comprises a first transducer section coupled with a hinge to a second transducer section to rotate about the hinge based on the curvature of the breast tissue.

15. The mammogram system of claim 13, wherein the flexible transducer assembly comprises a plurality of capacitive micromachined ultrasound transducers formed onto a flexible substrate, and the flexible substrate is a polymide formed using lithography.

16. The mammogram system of claim 13, wherein the flexible transducer assembly is configured to obtain data from a retroglandular area of the breast tissue.

17. A method for imaging comprising:
compressing breast tissue with a compression paddle having a membranous sheet to expose a chest wall of a patient; and
obtaining ultrasound image data of an area adjacent the chest wall of the patient.

18. The method of claim 17, wherein the membranous sheet is less than 50 microns in thickness.

19. The method of claim 17, wherein obtaining ultrasound image data of the area adjacent the chest wall of the patient includes obtaining a plurality of two-dimensional ultrasound images of the area adjacent the chest wall of the patient, and forming a three-dimensional image based on the plurality of two-dimensional images.

20. The method of claim 17, wherein obtaining ultrasound image data of the area adjacent the chest wall of the patient includes scanning the area adjacent the chest wall of the patient with a transducer that dynamically conforms to the breast tissue curvature.
